(19) United States
(12) Patent Application Publication
(10) Pub. No.: US 2018/0078724 A1
(43) Pub. Date: Mar. 22, 2018

(54) TRACHEOSTOMY TUBE PACKS, TUBE ASSEMBLIES, SETS AND METHODS

(71) Applicant: SMITHS MEDICAL INTERNATIONAL LIMITED, Ashford (GB)

(72) Inventors: Eric Pagan, Hythe (GB); Ian Douglas Stace, Minster-in-Thanet (GB); Neil Stephen Veasey, Ashford (GB)

(73) Assignee: SMITHS MEDICAL INTERNATIONAL LIMITED, Ashford (GB)

(21) Appl. No.: 15/569,436

(22) PCT Filed: Apr. 21, 2016
(86) PCT No.: PCT/GB2016/000089

§ 371 Date: Oct. 26, 2017

(30) Foreign Application Priority Data
May 16, 2015 (GB) 1508609.3

(51) Int. Cl. A61M 16/04

(52) U.S. Cl. CPC A61M 16/0465; A61M 16/0434; A61M 2209/06; A61M 2205/6036; A61M 16/0427; A61M 16/0402

(57) ABSTRACT

Tracheostomy tubes (1) of different sizes or of other different characteristics are marked on their flange (20) with a band (22) of differing colours or patterns. Similarly, inner cannulae (30) for use with tubes of specific sizes have a marking (33) of the same colour or pattern. The tube (1) and inner cannula (30) are contained in a sealed pack (40) having a tray (41) with a peel-off seal sheet (42) that also bears a marking (43) of the same colour or pattern. The sealed pack (40) is contained within an outer cardboard carton (50) having an end flap (51) which is sealed by a label (54) of the same colour or pattern so that tubes of the desired characteristic can be easily selected.

This invention relates to tracheostomy tube packs of the kind including an outer tracheostomy tube, an inner cannula adapted for removable insertion within the outer tube and packaging within which the outer tube and the inner cannula are enclosed.

Tracheostomy tubes are used to enable ventilation, respiration or spontaneous breathing of a patient. The tube is inserted into the trachea via a surgically-formed opening in the neck so that one end locates in the trachea and the other end locates outside the patient adjacent the neck surface. Tracheostomy tubes can be inserted by different techniques, such as the surgical cut down procedure carried out in an operating theatre or a cricothyroidotomy procedure, which may be carried out in emergency situations.

Tracheostomy tubes are generally used for more long-term ventilation or where it is not possible to insert an airway through the mouth or nose. The patient is often conscious while breathing through a tracheostomy tube, which may be open to atmosphere or connected by tubing to some form of ventilator. The tube is secured in position by means of a flange fixed with the machine end of the tube and positioned to extend outwardly on opposite sides of the tube. The anatomy of patients varies considerably according to age and build.

Tracheostomy tubes are often provided with a removable inner cannula. When secretions accumulate inside the assembly, the inner cannula can be removed and replaced. This avoids the need to replace the outer tube, which would be more traumatic and would have to be carried out by a doctor. Tracheostomy tubes are provided in a range of different characteristics for use in different situations. For example, the tubes can be made of different materials, different shapes, can be reinforced or unreinforced, can have a sealing cuff or be uncuffed or can be of a wide range of different sizes. Also, in a hospital, tubes may be available from different manufacturers. The outer tubes of different characteristics require different size inner cannulae. There is a problem, with conventional assemblies, in that there is a risk that a nurse or other practitioner could replace an inner cannula with a cannula of the wrong size or type. For example, a standard length inner cannula might be wrongly inserted in an extra length tube, or vice versa, or a non-fenestrated inner cannula could be wrongly used in a fenestrated tube, or vice versa. This could cause a poor fit or damage to the inner cannula or outer tube or could allow excessive mucus to build up that might obstruct breathing.

For this reason it is important to be able to identify and distinguish readily between different tubes and inner cannulae.

It is an object of the present invention to provide an alternative tracheostomy tube pack, set and method of making a pack.

According to one aspect of the present invention there is provided a tracheostomy tube pack of the above-specified kind, characterised in that the outer tracheostomy tube is provided with non-alphanumeric visible marking of a particular characteristic in a region of the tube visible externally of the patient when the tube is inserted in a patient and that is indicative of a property of the tube, and that the inner cannula and the packaging also are both provided with a corresponding marking of the same characteristic as that on the tube.

The property of the tube could be its size. The characteristic of the visible marking could be its colour or pattern. The region of the tube on which the marking is provided is preferably a flange of the tube. The packaging may include a tray with a peel-off sealing sheet. The packaging may include an outer carton in which the tray is contained.

According to another aspect of the present invention there is provided a tracheostomy tube assembly including an outer tracheostomy tube and an inner cannula adapted for removable insertion within the outer tube, characterised in that the outer tracheostomy tube is provided with a non-alphanumeric visible marking of a particular characteristic in a region of the tube visible externally of the patient when the tube is inserted in a patient and that is indicative of a property of the tube, and that the inner cannula is also provided with a corresponding marking of the same characteristic as that on the tube.

The property of the tube may be its size. The region of the tube on which the marking is provided may be a flange of the tube.

According to a further aspect of the present invention there is provided a set of a plurality of tracheostomy tube assembly packs each comprising packaging containing an outer tracheostomy tube and an inner cannula for use with the outer tube, at least two of the packs in the set containing tracheostomy tubes that differ in size between the packs, characterised in that the packaging, outer tracheostomy tube and inner cannula in each pack are each provided with non-alphanumeric visible marking of the same particular characteristic and that the marking on tubes of the same size are of the same characteristic whereas the marking on tubes of different sizes are of different characteristics.

The markings of tubes of different sizes may be of different colours or of different patterns.

According to yet another aspect of the present invention there is provided a method of making a tracheostomy tube pack including the steps of forming a tracheostomy tube having a non-alphanumeric visible marking of a particular characteristic on a part of the tube visible externally of the patient when the tube is inserted in a patient, forming an inner cannula dimensioned to fit within the tracheostomy tube and having a marking of the same characteristic as the tracheostomy tube, providing packaging also having a marking of the same characteristic as the tracheostomy tube and inner cannula and placing the tracheostomy tube and inner cannula in the packaging to complete the pack so that the nature of the tube and inner cannula in the pack can be determined from the marking on the pack.

A tracheostomy pack, a set of such packs and a method of making a pack will now be described, by way of example, with reference to the accompanying drawings in which:

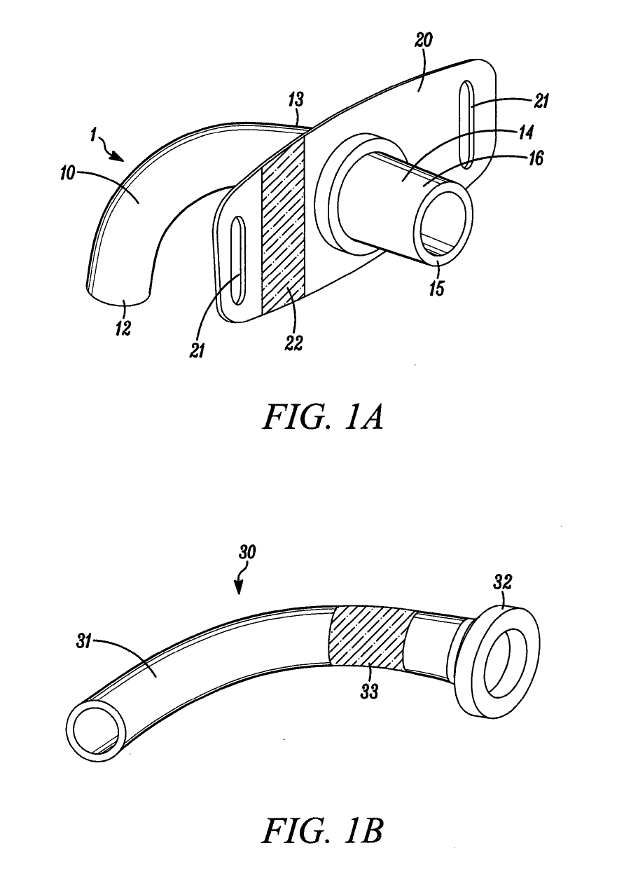

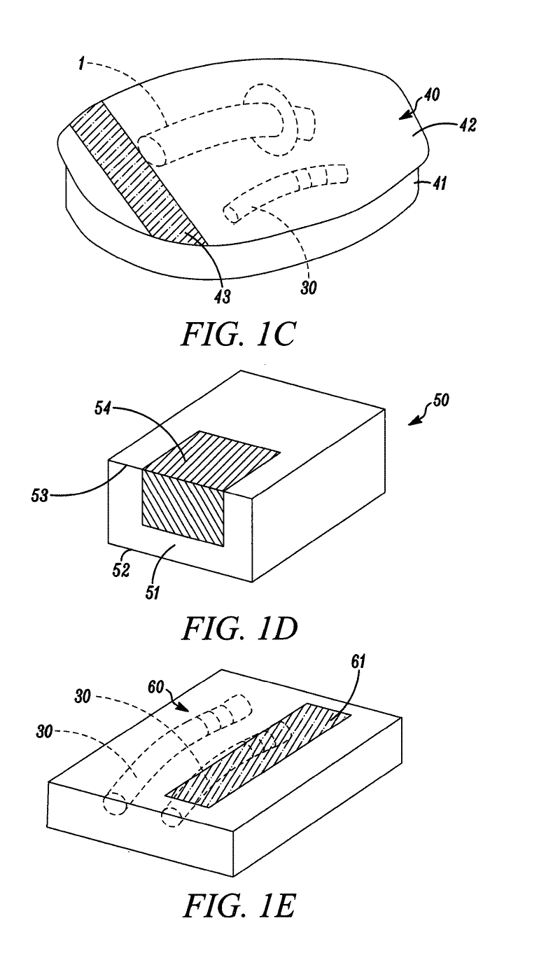

FIG. 1A is a perspective view of a tracheostomy tube of the kind included in the pack;

FIG. 1B is a perspective view of an inner cannula of the kind included in the pack;

FIG. 1C is a perspective view of a pack containing the tube and inner cannula are contained;

FIG. 1D is a perspective view an outer carton containing the pack;

FIG. 1E is a perspective view of a pack containing inner cannulae only; and

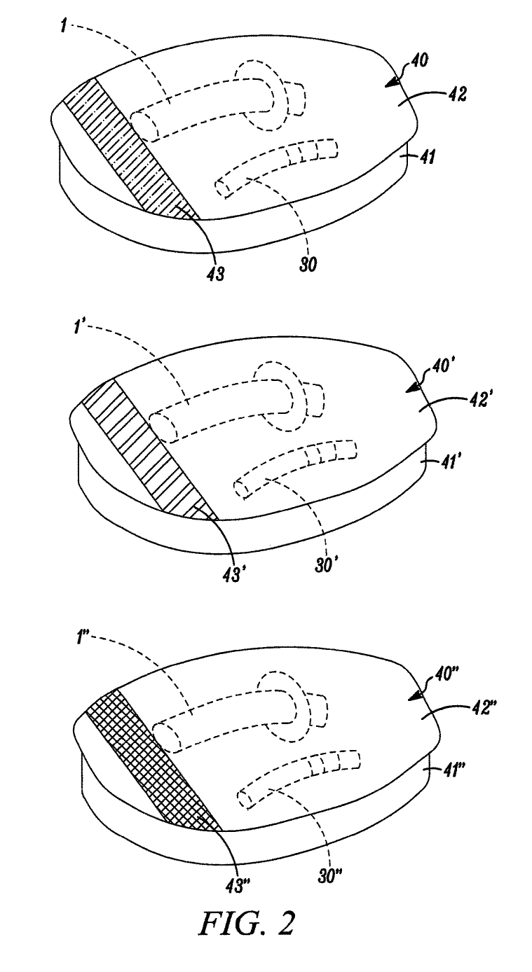

FIG. 2 is a perspective view of a set of three packs containing tracheostomy tubes and inner cannulae of three different sizes.

With reference first to FIG. 1A the outer tracheostomy tube 1 has a curved shaft 10 of circular section with a patient end 12 adapted to be located within the trachea of the patient. The shaft 10 may have a conventional sealing cuff towards its patient end although the tube shown does not have any sealing cuff. The shaft 10 is moulded or extruded and is bendable but relatively stiff, being made of a plastics material such as PVC or silicone. The machine end 13 of the shaft 10 is adapted, during use, to be located externally and adjacent the tracheostomy opening formed in the patient's neck. The machine end 13 of the shaft 10 supports a hub 14 and a 15mm male tapered connector 15 fixed with or forming a part of the hub and having a tapered external surface 16. The connector 15 is adapted to make a removable push fit in a conventional 15mm female connector (not shown) at one end of a breathing tube extending to a ventilator or anaesthetic machine. Alternatively, the machine end of the tube could be left open to atmosphere when the patient is breathing spontaneously. The tracheostomy tube 1 also includes a radially-extending support flange 20 that is adapted to lie against the skin surface of the neck on either side of the tracheostomy stoma. The flange 20 has openings 21 at opposite ends for attachment to a neck strap (not shown) used to support the tube with the patient's neck.

The flange 20 bears a marking of a particular characteristic on its outer surface in the form of a vertical, coloured band 22. The marking need not be on the flange 20 but could instead be on some other part of the tube 1 providing that it was clearly visible externally of the patient when the tube was inserted within a patient. The marking need not be a coloured band providing that it were in the form of a non-alphanumeric visual marking and could, for example, be patterned or otherwise visibly marked. The colour of the band 22 is indicative of a property of the tube 1, such as its size, more particularly, its inner diameter.

FIG. 1B shows an inner cannula 30 for use in the tracheostomy tube 1, the cannula having a curved shaft 31 with a hub 32 at its machine end. The cannula 30 is insertable into and removable from the tracheostomy tube 1. The shaft 31 of the inner cannula 30 bears a marking visible externally of the same characteristic (that is, the same colour) as that on the tube 1, in the form of an annular band 33 around the shaft close to the machine end hub 32.

The outer tracheostomy tube 1 and the inner cannula 30 are both contained in a sealed pack 40, shown in FIG. 1C, in the form of a base tray 41 moulded of a plastics material sealed across its upper surface by a peel-off, gas-permeable non-woven sheet 42 enabling gas sterilisation. The pack 40 may also contain other items such as a neck strap, wipes and the like (not shown). The pack 40 is marked in the same way as the tube 1 and inner cannula 30 by a coloured band 43, of the same colour as those on the tube and inner cannula, extending across the width of the pack.

The pack 40 is made by selecting a tracheostomy outer tube 1 of the desired size bearing the appropriate flange marking 22 and selecting a corresponding inner cannula 30 dimensioned to fit within the tracheostomy tube and having a marking 33 of the same colour as on the tracheostomy tube. The tube and cannula are placed in the base tray 41, covered by the permeable sheet 42 bearing the appropriately coloured band 43 for the size of tube so that the size of the tube and inner cannula in the pack can be readily determined from the colour of the band. The sheet 42 is then sealed about the tray 41. The sheet 42 would also typically bear a printed legend (not shown) indicating the outer diameter of the tube and other information conventionally given, or this could be on a separate label attached to the pack 40. The sealed pack 40 is placed in an outer carton 50 of the kind shown in FIG. 1D, which is described below, and subjected to gas sterilisation.

The outer cardboard carton 50 has a rectangular section and an end flap 51 hinged along its lower edge 52. The upper, free edge 53 of the flap 51 is tucked into the carton and is held in place by an adhesive label 54 marked with the same visual characteristics (that is, the same colour) as the bands 22, 33 and 43 on the tube 1, inner cannula 30 and pack 40. It serves to protect the pack within the carton.

With reference now also to FIG. 1E, each tracheostomy tube 1 is commonly used with several inner cannulae 30 since these are removed when secretions build up and are disposed of, to be replaced by a new inner cannula. Packs 60 containing only two or more inner cannulae 30 can, therefore, be provided bearing the characteristic coloured marking 61, such as, on its label, for the appropriate size tracheostomy tube.

FIG. 2 shows a set of three different packs 40, 40′ and 40″ containing tracheostomy tubes of three different sizes. Pack 40 contains the tube 1 and inner cannula 30 described earlier, the tube having a first size indicated by the colour of the band 43. Packs 40′ and 40″ contain tubes 1′ and 1″ and inner cannulae 30′ and 30″of the same kind but of different sizes. The coloured bands 43, 43′, 43″ on the packs 40, 40′ and 40″ enable the clinician readily to select the tube of the desired size to suit the build of the patient in which it is to be inserted. The coloured labels on the packs or cartons 50 enable staff who are stock-taking rapidly to assess tube sizes that need restocking and enable tubes of the correct size to be selected more easily.

The markings on the tube and other components need not be indicative of size but could be indicative of other properties of the tube, such as, for example, whether the tube is fenestrated, reinforced, of extra length or cuffed.

1-14. (canceled)

15. A tracheostomy tube pack including an outer tracheostomy tube, an inner cannula adapted for removable insertion within the outer tube and packaging within which the outer tube and the inner cannula are enclosed, characterised in that the outer tracheostomy tube is provided with non-alphanumeric visible marking of a particular characteristic in a region of the tube visible externally of the patient when the tube is inserted in a patient and that is indicative of a property of the tube, and that the inner cannula and the packaging also are both provided with a corresponding marking of the same characteristic as that on the tube.

16. A pack according to claim 15, characterised in that the property of the tube is its size.

17. A pack according to claim 15, characterised in that the characteristic of the visible marking is its colour.

18. A pack according to claim 16, characterised in that the characteristic of the visible marking is its pattern.

19. A pack according to claim 15, characterised in that the region of the tube on which the marking is provided is a flange of the tube.

20. A pack according to claim 15, characterised in that the packaging includes a tray with a peel-off sealing sheet.

21. A pack according to claim 20, characterised in that the packaging includes an outer carton in which the tray is contained.

22. A tracheostomy tube assembly including an outer tracheostomy tube and an inner cannula adapted for removable insertion within the outer tube, characterised in that the outer tracheostomy tube is provided with a non-alphanumeric visible marking of a particular characteristic in a region of the tube visible externally of the patient when the tube is inserted in a patient and that is indicative of a property of the tube, and that the inner cannula is also provided with a corresponding marking of the same characteristic as that on the tube.

23. A tracheostomy tube assembly according to claim 22, characterised in that the property of the tube is its size.

24. A tracheostomy tube assembly according to claim 22, characterised in that the region of the tube on which the marking is provided is a flange of the tube.

25. A set of a plurality of tracheostomy tube assembly packs each comprising packaging containing an outer tracheostomy tube and an inner cannula for use with the outer tube, at least two of the packs in the set containing tracheostomy tubes that differ in size between the packs, characterised in that the packaging , outer tracheostomy tube and inner cannula in each pack are each provided with non-alphanumeric visible marking of the same particular characteristic and that the marking on tubes of the same size are of the same characteristic whereas the marking on tubes of different sizes are of different characteristics.

26. A set of packs according to claim 25, characterised in that the markings of tubes of different sizes are of different colours.

27. A set of packs according to claim 25, characterised in that the markings of tubes of different colours are of different patterns.

28. A method of making a tracheostomy tube pack including the steps of forming a tracheostomy tube having a non-alphanumeric visible marking of a particular characteristic on a part of the tube visible externally of the patient when the tube is inserted in a patient, forming an inner cannula dimensioned to fit within the tracheostomy tube and having a marking of the same characteristic as the tracheostomy tube, providing packaging also having a marking of the same characteristic as the tracheostomy tube and inner cannula and placing the tracheostomy tube and inner cannula in the packaging to complete the pack so that the nature of the tube and inner cannula in the pack can be determined from the marking on the pack.
